(19) United States
(12) Patent Application Publication
(10) Pub. No.: US 2023/0149410 A1
(43) Pub. Date: May 18, 2023

(54) TREATMENT OF CONDITIONS ASSOCIATED WITH THYROID HORMONE

(71) Applicant: Linzy O. SCOTT, III, Atlanta, GA (US)

(72) Inventor: Linzy O. SCOTT, III, Atlanta, GA (US)

(21) Appl. No.: 17/996,865

(22) PCT Filed: Apr. 22, 2021
(86) PCT No.: PCT/US2021/028675

§ 371 Date: Oct. 21, 2022

Related U.S. Application Data
(60) Provisional application No. 63/013,960, filed on Apr. 22, 2020.
(60) Provisional application No. 63/088,523, filed on Oct. 7, 2020.
(60) Provisional application No. 63/135,118, filed on Jan. 8, 2021.

(51) Int. Cl. A61K 31/519; A61K 31/192; A61K 45/06; A61P 5/14

(52) U.S. Cl. CPC A61K 31/519; A61K 31/192; A61K 45/06; A61P 5/14

(57) ABSTRACT

Methods and compositions for treating and preventing medical conditions associated with the distribution, transport, and deiodination of thyroid hormone in the central nervous system (“CNS”), including but not limited to the brain and spinal cord are disclosed. In particular, methods and compositions for treating Allan Herndon Dudley Syndrome are disclosed.

### CROSS REFERENCE TO RELATED APPLICATIONS

This application claims the benefit of priority to U.S. Provisional Application 63/013,960, filed Apr. 22, 2020, U.S. Provisional Application 63/088,523, filed Oct. 7, 2020, and U.S. Provisional Application 63/135,118, filed Jan. 8, 2021, each of which are incorporated by reference herein in their entireties.

### FIELD

The subject matter disclosed herein generally relates to methods and compositions for diagnosing, treating, and preventing medical conditions associated with the distribution, transport, and deiodination of thyroid hormone in the central nervous system (“CNS”), including but not limited to the brain and spinal cord. In a more specific aspect, the subject matter disclosed herein relates to methods and compositions for treating Allan Herndon Dudley Syndrome.

### BACKGROUND

Allan Herndon Dudley Syndrome (“AHDS”) was described in 1944. However, AHDS was not linked to thyroid hormone transport issues until approximately 2004, when it was found that AHDS is caused by a mutation with respect to monocarboxylate transporter 8 (“MCT8”). MCT8 is encoded by the SLC16A2 gene, and MCT8 transports a variety of iodothyronines, including thyroid hormones T3 and T4. Notwithstanding this mutation, affected individuals (who are usually males given that this is an X-linked mutation with females serving as carriers) typically have normal thyroid hormone in the “blood” through testing of thyroid hormone levels in the plasma or serum. In other words, they can be euthyroid in the blood, even without receiving any thyroid hormone medication, or they can display a thyroid panel in the blood representative or consistent with a hyperthyroid clinical picture. Their thyroid gland is able to produce thyroid hormone, but because of the genetic mutation, transport of thyroid hormone is affected and they have profound hypothyroidism in the CNS, for the most part in neurons at an intracellular level. Thus, individuals with AHDS present with a confounding clinical picture, whereby such individuals present with hyperthyroidism or euthyroidism in the blood, but profound hypothyroidism in the CNS.

MCT8 is strongly expressed in the CNS. As a result, specifically T3 is not properly transported in the CNS (primarily with respect to neurons), resulting in profound hypothyroidism intracellularly in brain tissue. Thus, an individual with AHDS has profound hypothyroidism in the brain, but relative hyperthyroidism or euthyroidism in the blood. As a result of AHDS, these individuals have debilitating conditions. They can present relatively normal at birth, but within the ensuing months signs of hypotonia can appear. Then, the individuals become progressively worse, with severe intellectual disability and debilitating movement disorders, including dystonia and spasticity. At present, there is no treatment for these AHDS individuals. There is no cure. It is also a relatively rare disease that is estimated to affect upwards of approximately 50,000 individuals worldwide, while currently there are less than 500 known cases. What is thus needed are methods and compositions that can treat AHDS (MCT8 deficiency) and/or alleviate one or more of its symptoms. The compositions and methods disclosed herein address these and other needs.

### SUMMARY

In accordance with the purposes of the disclosed materials and methods, as embodied and broadly described herein, the disclosed subject matter, in one aspect, relates to compounds, compositions and methods of making and using compounds and compositions. In specific aspects, the disclosed subject matter relates to therapies for treating MCT8 deficiency (Allan Herndon Dudley Syndrome or AHDS) and/or alleviating one or more of its symptoms, as well as treating conditions associated with AHDS.

In specific examples, the disclosed subject matter relates to methods and compositions for preventing and/or treating people with AHDS from developing problems associated with folate deficiencies. In some embodiments, the disclosed subject matter provides a method of administering folate to people with AHDS.

In some embodiments, the disclosed subject matter provides a method of administering a pharmaceutical composition or nutritional supplement comprising one or more of a folate, vitamin B12, vitamin B6, or vitamin B2 to people with AHDS, as such vitamins play intricate roles in connection with the metabolism of folate. In some embodiments, the disclosed subject matter further provides a method of administering a reduced folate to individuals with AHDS. In some embodiments, the disclosed subject matter further provides a method of administering one or more of a reduced folate, vitamin B12, vitamin B6, or vitamin B2 to individuals with AHDS. Yet in another embodiment, the disclosed subject matter further provides a method of administering one or more of folinic acid, vitamin B12, vitamin B6, or vitamin B2 to individuals with AHDS. Yet in another embodiment, the disclosed subject matter further provides a method of administering one or more of 5-methyltetrahydrofolate, 5-methyltetetrahydrofolic acid, vitamin B12, vitamin B6, or vitamin B2 to individuals with AHDS. And in some embodiments the administration of a pharmaceutical composition or nutritional supplement comprising one or more of a folate, vitamin B12, vitamin B6, or vitamin B2 will treat or prevent related deficiencies in the CNS, in particular with respect to cells in brain tissue. In some embodiments, the disclosed subject matter includes the administration of a pharmaceutical composition or nutritional supplement comprising one or more of a folate, vitamin B12, vitamin B6, or vitamin B2 and can be coupled with the administration of one or more of the Other Elements and/or one or more of the other vitamins of the Vitamin B Complex. In some embodiments, the disclosed subject matter includes the administration of a pharmaceutical composition or nutritional supplement comprising one or more of the Other Elements and/or one or more of the other vitamins of the Vitamin B Complex.

In connection with the treatment of AHDS, in order to overcome the reduced ability to transport thyroid hormone into the cells of brain tissue as a result of an MCT8 related mutation, practitioners have resorted to the utilization of various thyroid hormone analogs, thyroid hormone chaperones, thyroid hormone thyromimetics, or thyroid hormone gene therapy as a means of promoting T3 in the cells of brain tissue. However, as noted herein, such therapies do not sufficiently address related folate deficiencies, or deficiencies in metabolites related to folate. The following embodiments facilitate prevention and treatment of such deficiencies for individuals with AHDS. In addition, in connection with the treatment of AHDS, in order to address the hyperthyroidism that occurs in the blood outside of the CNS, and to address the hypothyroidism that occurs in brain tissue, practitioners have resorted to the utilization of anti-thyroid drugs and/or thyroid hormone drugs as a means of reducing T3 levels in the blood outside of the CNS and promoting the production of thyroid hormone in brain tissue. However, as noted herein, such therapies do not sufficiently address related folate deficiencies, or deficiencies in metabolites related to folate. The following embodiments also facilitate prevention and treatment of such deficiencies for individuals with AHDS.

In some embodiments, the disclosed subject matter provides a method of administering a thyroid hormone analog with a pharmaceutical composition or nutritional supplement comprising one or more of a folate, vitamin B12, vitamin B6, or vitamin B2. In other embodiments, in lieu of a pharmaceutical composition or nutritional supplement comprising one or more of a folate, vitamin B12, vitamin B6, or vitamin B2, or in addition to a pharmaceutical composition or nutritional supplement comprising one or more of a folate, vitamin B12, vitamin B6, or vitamin B2, the administration may additionally include one or more of the Other Elements and/or one or more of the other vitamins of the Vitamin B Complex.

In some embodiments, the disclosed subject matter provides a method of administering a thyroid hormone chaperone with a pharmaceutical composition or nutritional supplement comprising one or more of a folate, vitamin B12, vitamin B6, or vitamin B2. In other embodiments, in lieu of a pharmaceutical composition or nutritional supplement comprising one or more of a folate, vitamin B12, vitamin B6, or vitamin B2, or in addition to a pharmaceutical composition or nutritional supplement comprising one or more of a folate, vitamin B12, vitamin B6, or vitamin B2, the administration may additionally include one or more of the Other Elements and/or one or more of the other vitamins of the Vitamin B Complex.

In some embodiments, the disclosed subject matter provides a method of administering a thyroid hormone thyromimetic with a pharmaceutical composition or nutritional supplement comprising one or more of a folate, vitamin B12, vitamin B6, or vitamin B2. In other embodiments, in lieu of a pharmaceutical composition or nutritional supplement comprising one or more of a folate, vitamin B12, vitamin B6, or vitamin B2, or in addition to a pharmaceutical composition or nutritional supplement comprising one or more of a folate, vitamin B12, vitamin B6, or vitamin B2, the administration may additionally include one or more of the Other Elements and/or one or more of the other vitamins of the Vitamin B Complex.

In some embodiments, the disclosed subject matter provides a method of administering a thyroid hormone gene therapy with a pharmaceutical composition or nutritional supplement comprising one or more of a folate, vitamin B12, vitamin B6, or vitamin B2. In other embodiments, in lieu of a pharmaceutical composition or nutritional supplement comprising one or more of a folate, vitamin B12, vitamin B6, or vitamin B2, or in addition to a pharmaceutical composition or nutritional supplement comprising one or more of a folate, vitamin B12, vitamin B6, or vitamin B2, the administration may additionally include one or more of the Other Elements and/or one or more of the other vitamins of the Vitamin B Complex.

In some embodiments, in connection with the treatment of AHDS, the disclosed subject matter provides a method of administering an anti-thyroid drug with a pharmaceutical composition or nutritional supplement comprising one or more of a folate, vitamin B12, vitamin B6, or vitamin B2. In other embodiments, in lieu of a pharmaceutical composition or nutritional supplement comprising one or more of a folate, vitamin B12, vitamin B6, or vitamin B2, or in addition to a pharmaceutical composition or nutritional supplement comprising one or more of a folate, vitamin B12, vitamin B6, or vitamin B2, the administration may additionally include one or more of the Other Elements and/or one or more of the other vitamins of the Vitamin B Complex.

In some embodiments, in connection with the treatment of AHDS, the disclosed subject matter provides a method of administering a thyroid hormone drug with a pharmaceutical composition or nutritional supplement comprising one or more of a folate, vitamin B12, vitamin B6, or vitamin B2. In other embodiments, in lieu of a pharmaceutical composition or nutritional supplement comprising one or more of a folate, vitamin B12, vitamin B6, or vitamin B2, or in addition to a pharmaceutical composition or nutritional supplement comprising one or more of a folate, vitamin B12, vitamin B6, or vitamin B2, the administration may additionally include one or more of the Other Elements and/or one or more of the other vitamins of the Vitamin B Complex.

In some embodiments, the disclosed subject matter provides a composition of a thyroid hormone analog and one or more of a folate, vitamin B12, vitamin B6, or vitamin B2. In other embodiments, in lieu of one or more of a folate, vitamin B12, vitamin B6, or vitamin B2, or in addition to one or more of a folate, vitamin B12, vitamin B6, or vitamin B2, the composition may additionally include one or more of the Other Elements and/or one or more of the other vitamins of the Vitamin B Complex.

In some embodiments, the disclosed subject matter provides a composition of a thyroid hormone chaperone and one or more of a folate, vitamin B12, vitamin B6, or vitamin B2. In other embodiments, in lieu of one or more of a folate, vitamin B12, vitamin B6, or vitamin B2, or in addition to one or more of a folate, vitamin B12, vitamin B6, or vitamin B2, the composition may additionally include one or more of the Other Elements and/or one or more of the other vitamins of the Vitamin B Complex.

In some embodiments, the disclosed subject matter provides a composition of a thyroid hormone thyromimetic and one or more of a folate, vitamin B12, vitamin B6, or vitamin B2. In other embodiments, in lieu of one or more of a folate, vitamin B12, vitamin B6, or vitamin B2, or in addition to one or more of a folate, vitamin B12, vitamin B6, or vitamin B2, the composition may additionally include one or more of the Other Elements and/or one or more of the other vitamins of the Vitamin B Complex.

In some embodiments, the disclosed subject matter provides a composition of a thyroid hormone gene therapy and one or more of a folate, vitamin B12, vitamin B6, or vitamin B2. In other embodiments, in lieu of one or more of a folate, vitamin B12, vitamin B6, or vitamin B2, or in addition to one or more of a folate, vitamin B12, vitamin B6, or vitamin B2, the composition may additionally include one or more of the Other Elements and/or one or more of the other vitamins of the Vitamin B Complex.

In another embodiment, the disclosed subject matter provides a composition of an anti-thyroid drug and one or more of the Other Elements and/or one or more of the vitamins of the Vitamin B Complex.

In another embodiment, the disclosed subject matter provides a composition of a thyroid hormone drug and one or more of the Other Elements and/or one or more of the vitamins of the Vitamin B Complex.

In a preferred embodiment, the methods and compositions for prevention and treatment of conditions associated with AHDS comprise 5-methyltetrahydrofolic acid or 5-methyltetrahydrofolate. In another preferred embodiment, the composition for prevention and treatment of conditions associated with AHDS comprise 5-methyltetrahydrofolic acid or 5-methyltetrahydrofolate, either alone or with either a thyroid hormone analog, a thyroid hormone chaperone, a thyroid hormone thyromimetic, or a thyroid hormone gene therapy. In another preferred embodiment, the composition of 5-methyltetrahydrofolic acid or 5-methyltetrahydrofolate and either a thyroid hormone analog, thyroid hormone chaperone, thyroid hormone thyromimetic, or thyroid hormone gene therapy, also comprise one or more of the Other Elements and/or one or more of the other vitamins of the Vitamin B Complex.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION

The materials, compounds, compositions, and methods described herein may be understood more readily by reference to the following detailed description of specific aspects of the disclosed subject matter, and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, publication(s) may be referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise.

“Optional” or “optionally” means that the subsequently described event or circumstance can or cannot occur, and that the description includes instances where the event or circumstance occurs and instances where it does not.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

By “prevent” or other forms of the word, such as “preventing” or “preventative” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “treatment” refers to obtaining beneficial or desired clinical results. Beneficial or desired clinical results include, but are not limited to, any one or more of: alleviation of one or more symptoms (such as neurological damage), diminishment of extent of neurological damage, or stabilized (i.e., not worsening) state of neurological damage.

The term “individual” preferably refers to a human in need of treatment with a composition as disclosed herein. However, the term “individual” can also refer to non-human animals, preferably mammals such as dogs, cats, horses, cows, pigs, sheep and non-human primates, among others, that are in need of treatment with the compositions disclosed herein. “Individual” is used interchangeably with “patient” herein.

As used herein, “folate(s)” refers to a group of pteroylglutamate acids that become structurally and functionally altered when reduced as well as their reduced products. Thus term “folate” refers to folic acid as well as any derivative thereof. Folic acid, (N-[4-(2-Amino-3,4-dihydro-4-oxo-6-pteridinylmethylamino)-benzoyl]-L-glutamic acid) also known as vitamin B9 or folicin as well as N-pteroyl-L-glutamic acid and N-pteroyl-L-glutamate, is a non-reduced folate. In humans, folates are absorbed most readily as the most active form 6(R,S)-5-methyltetrahydrofolate (6(S) methyltetrahydrofolate being the most biologically active) and it is the principal circulating form of folate (referred to herein as “reduced folate”). A nonexclusive list of other reduced folates (also included in the definition of “reduced folates”) are 10-methylenetetrahydrofolate, 10-formyltetrahydrofolic acid, 5-formyltetrahydrofolic acid, 5-forminino tetrahydrofolic acid, 5,10-methenyltetrahydrofolic acid, 5,10-methyltetrahydrofolic acid, L-methylfolate, and 6(R,S)-5-formyltetrahydrofolate (folinic acid), and tetrahydrofolic acid/tetrahydrofolate. The term “folate” is used as a genus, and generally refers to any of these forms of folate: folic acid and any form of reduced folates, including 5-methyltetrahydrofolic acid. The term “folate” also includes the pharmaceutical products Isovorin (levofolinic acid and/or levofolinate), Wellcovorin, Leucovorin, Metafolin and Quatrefolic.

By “Vitamin B Complex” is meant any one or more of the following compounds, vitamin B1 (thiamine), Vitamin B2 (riboflavin), Vitamin B3 (niacin or niacinamide), Vitamin B5 (pantothenic acid), Vitamin B6 (pyridoxine, pyridoxal, pyridoxol, pyridoxamine, pyridoxamine phosphate, or pyridoxine hydrochloride), Vitamin B7 (biotin), Vitamin B9 (folic acid), Vitamin B12.

Vitamin B12, also called cobalamin, is a water-soluble vitamin. Vitamin B12 refers to a group of cobalt-containing vitamer compounds known as cobalamins: these include, but are not limited to, cyanocobalamin, hydroxocobalamin, and the two naturally occurring cofactor forms of B12 in the human body: δ′-deoxyadenosylcobalamin (adenosylcobalamin— AdoB12), the cofactor of Methylmalonyl Coenzyme A mutase (MUT), and methylcobalamin (MeB12), the cofactor of 5-methyltetrahydrofolate-homocysteine methyltransferase (MTR).

By the term “Other Elements” is meant one or more of the following compounds (for descriptive purposes as to why certain compounds in this list are included, several enzymes where the compounds are relevant are described in this list. Other compounds disclosed in the material and methods section are also included here as “Other Elements”): calcium, aconitic, alanine, alpha amino butyric acid, arginine, copper, cystine, ethanolamine, glutamate, glutamic acid, glutamine, glutaric, glycine, histidine, homocysteine, hydroxyproline, isoleucine, lactate, lactic acid, lysine, magnesium, methionine, 1-methylhistidine, 3-methylhistidine, phenylalanine, s-adenosyl-methionine, serine, threonine, tryptophan, tyrosine valine, zinc, L-carnitine, iron, vitamin D, flavin mononucleotide, flavin adenine dinucleotide, pyridoxal-5′-phosphate, s-adenosylmethionine, n-acetylcysteine, betaine, glucose, magnesium, cobalt, cadmium, and adenosine triphosphate, tryptophan 5-hydroxytryptophan, L-dopa, phenylalanine hydroxylase, tyrosine hydroxylase, tryptophan hydroxylase, aromatic L-amino decarboxylase, melatonin, monoamine oxidase, catechol-O-methyltransferase, 3-O-methyldopa, dopamine beta-hydroxylase, copper, phenylethanolamine, dihydroxyphenylacetic acid, 3-metyramine, normetanephrine, metanephrine, 3-methoxy-4-hydroxyphenylglycole, vanillmandelic acid, vanillactic acid, and in the biosynthesis of neurotransmitters (including serotonin, melatonin, dopamine, norepinephrine and epinephrine), as well as its implications in nitric oxide synthase and BH3 and BH2) (dihydrobiopterin), thymidylate synthase, deoxyuridine monophosphate, deoxythymidine monophosphate, choline, betaine, dimethylglycine, betaine-homocysteine methyltransferase, methionine, s-adenosyl-methionine (SAMe), S-adenosylhomocysteine hydrolase, S-adenosyl-homocysteine, S-adenosylmethionine, methionine-adenosyltransferase, 5-aminoimidazole-4-carboxamide ribonucleotide, adenosine triphosphate (AICAR), formyl-AICAR, homocysteine, cystathionine B-synthase, cystathionine, cystathionine y-lyase, taurine, sulphate⁺H₂O, or related to folate distributors or transporters or receptors or processes regulating intracellular folate concentrations or folate derivatives, including but not limited to, folate receptor alpha, proton-coupled folate transporter (PCFT), reduced folate carrier (RFC1) and RBC-1, FRB (also known as FR-2 or FOLR-2), ATP-dependent FR1-mediated endocystosis with PCFT, folylpoly-y-glutamate carboxypeptidase, polyglutamyl tetrahydrofolate, monoglutamyl tetrahydrofolate, folylpolyglutamate synthetase, folylpolyglutamate synthase, and y-glutamate hydrolase representing the lysosomal enzyme for folylpolyglutamate breakdown to monoglutamate vitamin D, molybdopterin, pyrroloquinone, quinone, semiquinone, hydroquinone, topaquinone, flavin-N(5)-oxide, pyrophosphate, iron, trimethylglycine, Cystadone, tyramine, pramipexole, ropinorole, rotigotine, MAO inhibitors (including, but not limited to, tranylcypromine, selegiline, and phenelzine), anticholinergic drugs (including, but not limited to, trihexyphenidyl, benztropine, and biperiden), benzodiazepines (including, but not limited to, clobazam and diazepam), and clonidine. In addition to the foregoing, with respect to folates, there can be blocking FR1 auto-antibodies, or genetic defects in FOLR1 genes, and other antibodies or genetic defects to all the different folate transporters, receptors, distributors or processes regulating intracellular folate concentrations. Additional items that can be observed or measured for diagnostic purposes, or that can be used as treatments pursuant to the methods and compositions set forth in this filing include alpha aminoadipic semialdehyde, glucose, lactate, lactic acid, pyruvate, sialic acid, succinyladenosine, glutaric acid, co-enzyme Q10, carnitine, creatine, thymidine/deoxyuridine, thymidine phosphorylase enzyme, sex hormone binding globulin, cholesterol, ADP, N-acetylcysteine, clemastine, ammonium, succinate dehydrogenase, cytochrome oxidase, citrate synthase, citric acid cycle, selenium, selenoproteins, cystatin, cystatin C, glial fibrillary acidic protein, neurofilament light protein, formylglycinamide ribonucleotide, glycinamide ribonucleotide, MTHFD1 (per HGNC approved name), MTHFS (per HGNC approved name), reduced form of glutathione (GSH), oxidized form of glutathione (GSSG), reactive oxygen species (ROS), glutathione, glutathione peroxidase, glutathione disulfide, glutathione reductase, glutamate, glutamine, glutamine synthase, mitochondrial glutaminase, α-ketoglutarate, gamma-amino butyric acid (GABA), zinc, 3-P-glycerate, 3-phosphoglycerate dehydrogenase, 3-P—OH-Pyruvate, 3-phosphohydroxypyruvate transimase, 3-P-serine, and 3-phosphoserine phosphatase.

The term “cerebrospinal folate deficiency” (also referred to as cerebral folate deficiency) is associated with decreased levels of 5-methyltetrahydrofolate in the cerebrospinal fluid (CSF). In some conditions, the decreased level of folate in CSF is also associated with normal folate levels in the plasma and red blood cells. The onset of symptoms caused by the deficiency of folates in the brain generally begin within the first year of life, but with respect to Example #2 referenced in the examples contained herein exhibited themselves at birth or within the immediate months thereafter. This is followed by delayed development, with deceleration of head growth, hypotonia, and ataxia, followed in many cases by dyskinesias (choreo-athetosis, hemiballismus), spasticity, and speech difficulties, as well as numerous other cognitive, social, behavioral, psychological and physical conditions.

The term “iron” as it relates to nutritional supplementation, refers to any form of iron that is generally known to supplement nutrition; for example, an iron (II) salt, an iron (III) salt, or carbonyl iron.

The term “anti-thyroid drug” is a drug, agent or medication directed against the thyroid gland for the purposes of reducing thyroid function. The anti-thyroid drugs include, but are not limited to, carbimazole, methimazole, potassium perchlorate, potassium iodide and propylthiouracil (PTU). These drugs are used to treat hyperthyroidism (overactivity of the thyroid gland) or conditions where there is excessive circulating thyroid hormone, primarily in order to reduce the excessive thyroid activity before surgery and to treat and maintain patients not having surgery and to otherwise address excessive circulating thyroid hormone levels.

The term “thyroid hormone drug” is a drug, agent, medication or hormone that acts as a replacement for a hormone that is normally produced by the thyroid gland to regulate the body's energy and metabolism. Thyroid hormone drugs include but are not limited to: Levothyroxine, Levothyroxine Sodium, Liothyronine Sodium, Liotrix, Thyroglobulin, Thyroid (for example, desiccated thyroid hormone), Thyroxine, and Triiodothyronine, and may be sold under the brand names Levoxyl, Synthroid, Levo-T, Unithroid, Levothroid, Levoxine, Levolet, Novothyrox, Triostat, Cytomel and Thyrolar. The definition of “thyroid hormone drug” also includes combinations of the foregoing, such as T4/T3 blends for instance, as well as slow release, controlled release, delayed release, or similar versions of the foregoing.

The term “thyroid hormone analog” relates to compounds that are similar in molecular structure with respect to thyroid hormone drugs, thereby allowing the thyroid hormone analog to have some degree of interaction with molecular targets of thyroid hormone drugs. Thyroid hormone analogs include but are not limited to: diiodothyropropionic acid (DITPA), tetraiodothyroacetic acid (TETRAC), and triiodothyroacetic acid (TRIAC or Tiratricol or Teatrois). Clemastine may be utilized in conjunction with thyroid hormone analogs.

The term “thyroid hormone chaperone” relates to a class of small molecule chemical chaperones whose function is to enhance the folding and/or the stability of proteins, and with respect to use in the treatment of AHDS, include but are not limited to: phenylbutyrate, 4-phenylbutyric acid, and sodium phenylbutyrate.

The term “thyroid hormone gene therapy” relates to the use of gene replacement therapy to transfer genetic material with regard to MCT8 into cells, and includes but is not limited to: adeno associated virus 9 based gene therapy (AAV9).

The term “thyroid hormone thyromimetic” relates to a compound that mimics the action of the thyroid gland or hormones produced by the thyroid gland, and include but are not limited to: eprotirome, sobetirome, and Sob-AM2.

Reference will now be made in detail to specific aspects of the disclosed materials, compounds, compositions, and methods, examples of which are illustrated in the accompanying Examples.

### Materials and Methods

Applicant has discovered the need for supplementation with one or more of a folate vitamin B2, B6, or B12, as well as all the other supplementation or treatment options disclosed herein, specifically including all other vitamins of the Vitamin B Complex (and their derivatives, including, but not limited to, 5-methyltetrahydrofolate, flavin mononucleotide, flavin adenine dinucleotide, FADH₂, and pyridoxal-5′-phosphate), serine, glycine, methionine, s-adenosylmethionine, n-acetylcysteine, betaine, glucose, zinc, magnesium, copper, cobalt, cadmium, and adenosine triphosphate, in individuals with AHDS or conditions associated with AHDS.

Previously, for example, WO 2011/006147 A1, which is incorporated by reference herein in its entirety, discloses circumstances where hypothyroidism occurred in a pregnant mother and her twin boys. The mother and twin boys suffered from profound hypothyroidism as evidenced by thyroid hormone testing in the plasma or serum of the body and not the CNS (thus the “blood”), and as evidenced by goiter in the twins in utero as well as immediately after birth. With respect to the twins, they were immediately treated with a thyroid hormone drug (T4) after birth, and after treatment, became chemically euthyroid as evidenced by thyroid hormone testing in the plasma or serum outside of the CNS (thus, the “blood”). However, notwithstanding becoming chemically euthyroid in the “blood,” Example #2 eventually displayed decreased 5-methyltetrahydrofolate in his cerebrospinal fluid, a neurological condition (outside of the “blood”). WO 2011/006147 A1 makes the discovery that the hypothyroidism found in the “blood” caused decreased folate in cerebrospinal fluid, and notwithstanding proper and standard of care treatment through the administration of thyroid hormone, thereby making the twins chemically euthyroid after birth, decreased folate in cerebrospinal fluid remained, even years after receiving proper and standard of care thyroid hormone treatment and being rendered chemically euthyroid in the blood. As a result, WO 2011/006147 A1 addresses the adverse outcome of being hypothyroid, but chemically euthyroid in the blood, while still having deficient folate in the cerebrospinal fluid, by using folate treatments for decreased folate in cerebrospinal fluid in individuals with hypothyroidism who have been treated with a thyroid medication and thereby have been rendered chemically euthyroid.

AHDS patients are different from the patients in WO 2011/006147 A1 in that this is not an individual with classic hypothyroidism in the blood, or an individual with hypothyroidism in the blood who is being classically treated with a thyroid hormone drug (for example T4) to render them chemically euthyroid in the blood. Rather, AHDS individuals have profound hypothyroidism in the brain, with a relative normal status in the blood or if anything, more of a hyperthyroid profile in the blood, while at the same time having extreme hypothyroidism in the brain or CNS. Applicant thus claims that folate, and preferably reduced folate, as well as vitamins B2, B6, and/or B12, as well as other metabolites disclosed herein, are treatments for AHDS individuals because AHDS individuals can suffer from decreased folate conditions in the brain and related deficiencies.

In addition, Applicant's compositions and methods for treating and diagnosing individuals who do not have AHDS, but who have problems in the distribution, transport or deiodination of thyroid hormones in the CNS (as in OATP1C1 defects), or issues with the distribution, transport, conversion or storage of folate (or any other B vitamins) throughout the CNS, and specifically in cells identified herein, or the ancillary issues associated with folate storage and metabolism, including vitamin B6 or vitamin B2 and all of their derivatives and cofactors, including flavin mononucleotide, flavin-adenine dinucleotide, FADH₂, and pyridoxal-5′-phosphate (also known as PLP), as well as other cofactors or other related metabolites in the applicable folate cycle/pathway, methionine cycle/pathway, and transsulphuration cycle/pathway, such as serine, glycine, methionine, s-adenosylmethionine, n-acetylcysteine, betaine, glucose, zinc, magnesium, copper, cobalt, cadmium, and adenosine triphosphate, can benefit from Applicant's methods and compositions disclosed herein. Such individuals may have hypothyroidism, and they have been rendered chemically euthyroid in the blood, but they still have hypothyroidism in the CNS due to distribution, transport or deiodination problems. In another situation, an individual may not have hypothyroidism in the blood, and may not have AHDS either, but they do have problems in the distribution, transport or deiodination of thyroid hormones specific to only the CNS or brain. These problems of distribution, transport or deiodination in all of the foregoing examples set forth above may be associated with genetic mutations or polymorphisms or single nucleotide polymorphisms (“SNPs”) in MCT8, as well as any other distributor or transporter or enzyme or protein involved in the distribution, transport or deiodination of thyroid hormone, including, but not limited to, albumin, transthyretin, thyroxine-binding globulin, monocarboxylate transporters (including MCT8 and MCT10), organic anion transporting polypeptides (including OATP1C1), 1-type amino acid transporters (including LAT1 and LAT2), and deiodinase enzymes (including D1, D2 and D3). In addition, the foregoing individuals described herein may have deficiencies or genetic mutations or polymorphisms or SNPs related to methylenetetrahydrofolate reductase (MTHFR) (such as C677T or A1298C), or deficiencies or genetic mutations or polymorphisms or SNPs related to flavin adenine dinucleotide (in hypothyroidism, there is a defective conversion of riboflavin to its flavin adenine dinucleotide co-enzyme affecting MTHFR), in flavin mononucleotide (thereby affecting the transsulphuration pathway) (including any and all SNPs, polymorphisms, mutations, or deletions related to riboflavin kinase (also known as flavokinase) or FAD synthase or any other enzymatic reactions occurring between the conversion of riboflavin to flavin mononucleotide to flavin adenine dinucleotide) or related to the folate (remethylation) cycle, or the methionine cycle or the transsulphuration cycle, or related to any metabolites, substances, or enzymes or other processes related to the foregoing cycles, including but not limited to dihydrofolate, dihydrofolate reductase, tetrahydrofolate, methylenetetrahydrofolate dehydrogenase/methenyltetrahydrofolate cyclohydrolase/formyltetrahydrofolate synthase, 5-formyltetrahydrofolate, 5-formimino tetrahydrofolate, 10-formyltetrahydrofolate, 5,10-methenyltetrahydrofolate, 5,10-methylenetetrahydrofolate, 5-methyltetrahydrofolate, any B vitamins of the B complex (including cobalamin and methylcobalamin (B12), B1 (thiamine), B2 (riboflavin), B3 (niacin), B-5 (pantothenic acid), B-6 (pyridoxine or pyridoxal-5′-phosphate or pyridoxamine phosphate), B7 (biotin) and all forms of folate (B9)), all metabolites, substances, cofactors and/or enzymes involved in the metabolism of vitamin B2, specifically riboflavin to flavin mononucleotide through riboflavin kinase and flavin mononucleotide to flavin adenine dinucleotide through FAD synthase with cofactors of zinc, magnesium, cobalt, cadium, and adenosine triphosphate, or in reverse enzymatic order, including through ectonucleotide pyrophosphate/phosphodiesterase family member 1 (using calcium as a cofactor) and low molecular weight phosphotyrosine protein phosphate), methionine synthase, methionine synthase reductase, serine, glycine, serine-hydroxymethyltransferase, purine synthesis, pterins and their derivatives, guanosine-5′-triphosphate, neopterin, biopterin (including tetrahydrobiopterin (BH4) and associated cofactor activities related to aromatic amino acid hydroxylase enzymes and use in the degradation of and metabolism of phenylalanine, tyrosine and tryptophan 5-hydroxytryptophan, L-dopa, phenylalanine hydroxylase, tyrosine hydroxylase, tryptophan hydroxylase, aromatic L-amino decarboxylase, melatonin, monoamine oxidase, catechol-O-methyltransferase, 3-O-methyldopa, dopamine beta-hydroxylase, copper, phenylethanolamine, dihydroxyphenylacetic acid, 3-metyramine, normetanephrine, metanephrine, 3-methoxy-4-hydroxyphenylglycole, vanillmandelic acid, vanillactic acid, and in the biosynthesis of neurotransmitters (including serotonin, melatonin, dopamine, norepinephrine and epinephrine), as well as its implications in nitric oxide synthase and BH3 and BH2) (dihydrobiopterin), thymidylate synthase, deoxyuridine monophosphate, deoxythymidine monophosphate, choline, betaine, dimethylglycine, betaine-homocysteine methyltransferase, methionine, s-adenosyl-methionine (SAMe), S-adenosylhomocysteine hydrolase, S-adenosyl-homocysteine, S-adenosylmethionine, methionine-adenosyltransferase, 5-aminoimidazole-4-carboxamide ribonucleotide, adenosine triphosphate (AICAR), formyl-AICAR, homocysteine, cystathionine B-synthase, cystathionine, cystathionine y-lyase, taurine, sulphate⁺H₂O, or related to folate distributors or transporters or receptors or processes regulating intracellular folate concentrations or folate derivatives, including but not limited to, folate receptor alpha, proton-coupled folate transporter (PCFT), reduced folate carrier (RFC1) and RBC-1, FRB (also known as FR-2 or FOLR-2), ATP-dependent FR1-mediated endocystosis with PCFT, folylpoly-y-glutamate carboxypeptidase, polyglutamyl tetrahydrofolate, monoglutamyl tetrahydrofolate, folylpolyglutamate synthetase, folylpolyglutamate synthase, and y-glutamate hydrolase representing the lysosomal enzyme for folylpolyglutamate breakdown to monoglutamate vitamin D, molybdopterin, pyrroloquinone, quinone, semiquinone, hydroquinone, topaquinone, flavin-N(5)-oxide, pyrophosphate, iron, trimethylglycine, cystadone, tyramine, pramipexole, ropinorole, rotigotine, MAO inhibitors (including, but not limited to, tranylcypromine, selegiline, and phenelzine), anticholinergic drugs (including, but not limited to, trihexyphenidyl, benztropine, and biperiden), benzodiazepines (including, but not limited to, clobazam and diazepam), and clonidine. In addition to the foregoing, with respect to folates, there can be blocking FR1 auto-antibodies, or genetic defects in FOLR1 genes, and other antibodies or genetic defects to all the different folate transporters, receptors, distributors or processes regulating intracellular folate concentrations. Additional items that can be observed or measured for diagnostic purposes, or that can be used as treatments pursuant to the methods and compositions set forth herein include alpha aminoadipic semialdehyde, glucose, lactate, lactic acid, pyruvate, sialic acid, succinyladenosine, glutaric acid, co-enzyme Q10, carnitine, creatine, thymidine/deoxyuridine, thymidine phosphorylase enzyme, sex hormone binding globulin, cholesterol, ADP, N-acetylcysteine, clemastine, ammonium, succinate dehydrogenase, cytochrome oxidase, citrate synthase, citric acid cycle, selenium, selenoproteins, cystatin, cystatin C, glial fibrillary acidic protein, neurofilament light protein, formylglycinamide ribonucleotide, glycinamide ribonucleotide, MTHFD1 (per HGNC approved name), MTHFS (per HGNC approved name), reduced form of glutathione (GSH), oxidized form of glutathione (GSSG), reactive oxygen species (ROS), glutathione, glutathione peroxidase, glutathione disulfide, glutathione reductase, glutamate, glutamine, glutamine synthase, mitochondrial glutaminase, a-ketoglutarate, gamma-amino butyric acid (GABA), zinc, 3-P-glycerate, 3-phosphoglycerate dehydrogenase, 3-P—OH-Pyruvate, 3-phosphohydroxypyruvate transimase, 3-P-serine, and 3-phosphoserine phosphatase.

As such, all of the foregoing can cause deficiencies in folate (and all of its derivatives) in the brain, in brain tissues, in the CNS, and intracellularly in the cell and disrupt the folate cycle, the transsulphuration pathway, and the methionine cycle, and all of their ancillary metabolites, substances, enzymes, and cofactors, and when this occurs in specific cells, the results thereof can be masked and difficult to diagnose and treat. For instance, with respect to folate in particular, this can all happen even if folate in cerebrospinal fluid is decreased or normal. Given that deficiencies in folate in the CNS and brain tissue can occur completely separate from deficiencies in cerebrospinal fluid, there is an additional need for the disclosed methods and compositions, with AHDS individuals serving as an example. AHDS individuals serve as an example where thyroid hormone, or the function of the thyroid gland, can be normal in the blood, but in the CNS, hypothyroidism that is isolated in the brain or CNS can cause folate issues. That also means that individuals who do not have AHDS can also have genetic mutations or polymorphisms or SNPs or antibodies or receptor issues or deficiencies implicated in the folate metabolic pathway that are specific to the CNS or brain, where otherwise they appear normal in the blood, such that they need Applicant's treatments, diagnostic methods, and methods and compositions for treatment. These deficiencies can occur in the cell and in brain tissue. Since they can occur in brain tissue, folate can be normal in the cerebrospinal fluid, but once folate leaves the cerebrospinal fluid and enters brain tissue, the intracellular issues can disrupt the folate cycle thereby causing decreased folate in the CNS outside of the cerebrospinal fluid. Therefore, the methods and compositions disclosed herein relate to treatment with respect to the cells and tissues within the brain and CNS, including but not limited to, glial cells, astrocytes, Bergmann glia, oligodendrocytes, oligodendrocyte progenitor cells, ependymal cells, tanycytes, microglia, radial glia, neurons, epithelial cells, stem cells, blood vessels, interneurons, pyramidal cells, Betz cells, motor neurons, Purkinje cells, and mast cells, Cajal-Retzius cells, and Schwann and including but not limited to the cerebrum, brainstem, cerebellum, cerebral cortex, neocortex, allocortex, frontal lobe, temporal lobe, parietal lobe, occipital lobe, central lobe, limbic lobe, insular lobe, precentral gyrus, postcentral gyrus, midbrain, pons, medulla oblongata, ventricular system, thalamus, epithalamus, pineal gland, hypothalamus, pituitary gland, subthalamus, limbic structures, amygdala, hippocampus, claustrum, basal ganglia, striatum, globus pallidus, substantia nigra, subthalamic nucleus, putamen, basal forebrain, circumventricular organs, dura mater, arachnoid mater, pia mater, glia limitans, nucleus accumbens, nucleus basalis, diagonal band of Broca, substantia innominate, medical septal nucleus, cerebellar tentorium, superior cerebellar peduncles, middle cerebellar penduncles, inferior cerebellar penduncles, clivus, foramen magnum, spinal cord, and choroid plexus.

What has previously been unknown is the relationship between folate levels intracellularly in the brain tissue of individuals with AHDS and thyroid hormone levels intracellularly in brain tissue of such individuals as a result of alterations in the transport of thyroid hormone, specifically T3, in the CNS. Individuals with AHDS appear either euthyroid or hyperthyroid in the blood outside of the CNS; however, AHDS individuals have profound hypothyroidism in cells in brain tissue, materially with respect to neuronal cells. Notwithstanding this profound hypothyroidism intracellularly in brain tissue, individuals with AHDS syndrome can have normal folate levels in the blood outside of the CNS, and even normal levels of folate in the cerebrospinal fluid, especially in their younger years of development. This clinical picture of euthyroidism or hyperthyroidism in the blood, combined with normal levels of folate in the blood outside of the CNS as well as normal levels of folate in the cerebrospinal fluid confound the clinical picture, as the profound hypothyroidism at the cellular level in brain tissue can go unobserved or undiagnosed. As a result, folate deficiencies and associated adverse outcomes involved in the Vitamin B Complex develop, with cascading effects on associated pathways leading to debilitating outcomes observed in individuals with AHDS. This surprising discovery has led to the present invention. Due to the over consumption of folate at the cellular level in brain tissue, over time in the later years of individuals with AHDS, signs of deficiencies of folate in the cerebrospinal fluid can then be observed.

Providing AHDS individuals with a pharmaceutical composition or nutritional supplement comprising one or more of a folate, vitamin B12, vitamin B6, or vitamin B2 can beneficially address and alleviate adverse outcomes associated with AHDS. In addition, certain Other Elements and other vitamins of the Vitamin B Complex as identified herein can also beneficially address and alleviate the adverse outcomes associated with AHDS. The present disclosure also addresses those who, during the course of treatment of AHDS, take a thyroid hormone analog, thyroid hormone chaperone, thyroid hormone thyromimetic, thyroid hormone gene therapy, anti-thyroid drug, or thyroid hormone drug. Supplementation with one or more of a folate, vitamin B12, vitamin B6, or vitamin B2 along with a thyroid hormone analog, thyroid hormone chaperone, thyroid hormone thyromimetic, thyroid hormone gene therapy, anti-thyroid drug, or thyroid hormone drug can provide a better means of preventing and/or treating folate deficiencies and the associated problems from such deficiencies. In addition, certain Other Elements and vitamins of the Vitamin B Complex as identified herein can also beneficially address and alleviate the adverse outcomes associated with AHDS in conjunction with the use of a thyroid hormone analog, thyroid hormone chaperone, thyroid hormone thyromimetic, thyroid hormone gene therapy, anti-thyroid drug, or thyroid hormone drug.

This invention can help prevent and further help diagnose the cause of folate and related deficiencies in individuals with AHDS, as leading researchers in the field of folate deficiencies as well as MCT8 deficiency are not focused on the deficiencies noted herein, but rather are focused on thyroid hormone analog, thyroid hormone chaperone, thyroid hormone thyromimetic, or thyroid hormone gene therapy that do not adequately address the deficiencies noted herein and the cascading effects and outcomes created thereby.

There is clearly a need to make the relationship between AHDS and brain tissue intracellular deficiencies in folate and related metabolites known so that they may be prevented and treated. The disclosed subject matter provides methods and compositions for prevention and treatment of conditions associated with AHDS and the metabolic deficiencies caused thereby. The invention is based on the discovery that in AHDS, individuals can appear euthyroid or hyperthyroid in the blood outside of the CNS, and as a result, the profound hypothyroidism that exists intracellularly in the CNS in brain tissue is unexpected and goes unobserved and untreated. Further, these individuals do not display abnormalities in folate in the blood outside in the CNS, and at least in some cases in their younger years deficiencies in folate in their cerebrospinal fluid can appear normal as well. Moreover, even when practitioners are aware that an individual suffers from AHDS due to an MCT8 deficiency, practitioners are still not aware of or focused on deficiencies in the CNS related to folate. Rather, these practitioners treat AHDS with a thyroid hormone analog, thyroid hormone chaperone, thyroid hormone thyromimetic, or thyroid hormone gene therapy which do not adequately address underlying folate and related deficiencies.

### Patient Population

Patients are those who suffer from AHDS, also known as MCT8 deficiency. In some additional embodiments, a patient can have a medical condition associated with the distribution, transport, and deiodination of thyroid hormone in the central nervous system (“CNS”), specifically including, but not limited to the brain and spinal cord.

### Pregnant Mothers and Fetuses

Those who are pregnant and are either a candidate to possibly deliver a child with an MCT8 deficiency (since mothers can be carriers and identified as individuals harboring the MCT8 mutation), or have already been identified as carrying a fetus with AHDS, are also suitable patients as disclosed herein, because treatment with a pharmaceutical composition or nutritional supplement comprising one or more of a folate, vitamin B12, vitamin B6, or vitamin B2 and/or one or more of the Other Elements disclosed herein and/or other vitamins of the Vitamin B Complex can be administered to the mother and transferred to the fetus via the placental barrier. Indeed, the debilitating conditions associated with AHDS can begin to develop in utero and the receipt of treatment by the AHDS fetus as early as possible is preferred.

### Fetus

Rather than treating the mother to effect placental transfer of the methods and compositions for the treatment of AHDS as disclosed herein, the methods and compositions of treatment disclosed herein may be directly administered to the fetus by any manner known in the art, and therefore, a fetus with AHDS is also the subject of this invention. Thus, in the methods disclosed herein the patient can be a fetus.

### Prevention Methods

While many of the uses of folate are generally well known, new conditions with respect to AHDS have been discovered that require the use of folates or other metabolites as noted herein that support the folate metabolic pathway. Therefore, it is the subject of this invention to disclose methods and compositions of administering a pharmaceutical composition or nutritional supplement comprising one or more of a folate, vitamin B12, vitamin B6, or vitamin B2 to those individuals suffering from AHDS, as well as certain Other Elements and/or other vitamins of the Vitamin B Complex.

Some of the first symptoms associated with AHDS are related to hypotonia. As the conditions progresses, there is developmental delay, spasticity, dystonia and severe intellectual disability. As conditions worsen, physical function is severely impaired. These are only a few of the conditions that may arise from AHDS. The methods and compositions discussed herein can prevent, or treat or alleviate the conditions associated with AHDS.

Administration of a pharmaceutical composition or nutritional supplement comprising one or more of a folate, vitamin B12, vitamin B6, or vitamin B2, as well as the Other Elements and the other vitamins of the Vitamin B Complex, can be done in any manner already known in the art in the treatment of AHDS. One embodiment is to administer a pharmaceutical composition or nutritional supplement comprising one or more of a folate, vitamin B12, vitamin B6, or vitamin B2 to people with AHDS. In a preferred embodiment, a reduced folate is administered to an individual with AHDS. A non-exclusive list of examples of reduced folates are: 10-formyltetrahydrofolic acid, 5-formyltetrahydrofolic acid, 5-forminino tetrahydrofolic acid, 5,10-methenyltetrahydrofolic acid, 5,10-methyltetrahydrofolic acid, 5-methyltetrahydrofolic acid, and 5-methyltetrahydrofolate.

The amount of folate administered by the methods and compositions of this invention will depend upon the size, age, and severity of the condition of the individual. Generally, from 30% to up to 5000% of the National Institutes of Health, Office of Dietary Supplements (NIH) generally recommended dosage guidelines will suffice. This is also true for the administration of vitamin B12, vitamin B6, or vitamin B2, as well as other vitamins of the Vitamin B Complex and the Other Elements. The amount of Vitamin B Complex and Other Elements are present in amounts that are from 30% to up to 5000% of the USFDA recommend daily guidelines.

In severe cases the amounts can be increased. Dosage amounts may need to be lower than NIH generally recommended dosage guidelines in the event of preventive measures, or in the event the individual is already taking supplements containing the foregoing, or in the event the individual is a premature infant or very newborn neonate.

In one embodiment, the amount of folate to be administered by the methods and compositions disclosed herein can be from about 0.2 mg to about 15 mg mg of folate per kg of weight (of the patient) per day. In other cases, higher dosages of folate at about 0.5 mg to about 3 mg/kg/day are required to normalize and address folate levels in the CNS. Yet, in other cases, where preventive measures are being taken, or when the individual is a fetus, premature newborn or term neonate, then dosage amounts can be lower than the foregoing.

In some embodiments, the amount of reduced folate to be administered by the methods and compositions disclosed herein can be from about 0.1 mg to about 1.0 mg of folate per kg of weight (of the individual) per day. In a preferred embodiment, the amount of reduced folate to be administered by the methods and compositions disclosed herein can be from about 0.5 mg to about 0.1 mg of folate per kg of weight (of the individual) per day. In other cases, higher dosages of folate at about 0.5 mg to about 3 mg/kg/day are required to normalize folate levels in the CNS. Yet, in other cases where preventive measures are being taken, or when the individual is a fetus, premature newborn or term neonate, then dosage amounts may be lower than the foregoing.

With respect to 5-methyltetrahydrofolate or 5-methyltetrahydrofolic acid, dosages can need to range from about 400 mcg to about 15 mg. In other cases, in severe deficiencies, the dosage amount may need to exceed 15 mg.

The following tables are provided by the NIH as the recommended dietary allowance for folate and other vitamins and minerals.

TABLE 1
Adequate Intake for Folate for Infants

| Age | Males and Females |
|---|---|
| (months) | (μg/day) |
| 0 to 6 | 65 |
| 7 to 12 | 80 |

TABLE 2
Recommended Dietary Allowances for
Folate for Children and Adults

| Age | Males and Females | Pregnant | Lactating |
|---|---|---|---|
| (years) | (μg/day) | (μg/day) | (μg/day) |
| 1 to 3 | 150 | N/A | N/A |
| 4 to 8 | 200 | N/A | N/A |
| 9 to 13 | 300 | N/A | N/A |
| 14 to 18 | 400 | 600 | 500 |
| 19+ | 400 | 600 | 500 |

TABLE 3
Recommended Dietary Allowances (RDAs) for Vitamin B12

| | | Males | Females | Pregnant | Lactating |
|---|---|---|---|---|---|
| Age | | (μg/day) | (μg/day) | (μg/day) | (μg/day) |
| Birth to 6 | months | 0.4 | 0.4 | | |
| 7 to 12 | months | 0.5 | 0.5 | | |
| 1 to 3 | years | 0.9 | 0.9 | | |
| 4 to 8 | years | 1.2 | 1.2 | | |
| 9 to 13 | years | 1.8 | 1.8 | | |
| 14+ | years | 2.4 | 2.4 | 2.6 | 2.8 |

TABLE 4
Recommended Dietary Allowances (RDAs) for Vitamin B6

| | | Male | Female | Pregnant | Lactating |
|---|---|---|---|---|---|
| Age | | (mg/day) | (mg/day) | (mg/day) | (mg/day) |
| Birth to 6 | months | 0.1 | 0.1 | | |
| 7-12 | months | 0.3 | 0.3 | | |
| 1-3 | years | 0.5 | 0.5 | | |
| 4-8 | years | 0.6 | 0.6 | | |
| 9-13 | years | 1.0 | 1.0 | | |
| 14-18 | years | 1.3 | 1.2 | 1.9 | 2.0 |
| 19-50 | years | 1.3 | 1.3 | 1.9 | 2.0 |
| 51+ | years | 1.7 | 1.5 | | |

TABLE 5
Recommended Dietary Allowances (RDAs)
for Riboflavin (Vitamin B2)

| | | Male | Female | Pregnant | Lactating |
|---|---|---|---|---|---|
| Age | | (mg/day) | (mg/day) | (mg/day) | (mg/day) |
| Birth to 6 | months* | 0.3 | 0.3 | | |
| 7-12 | months* | 0.4 | 0.4 | | |
| 1-3 | years | 0.5 | 0.5 | | |
| 4-8 | years | 0.6 | 0.6 | | |
| 9-13 | years | 0.9 | 0.9 | | |
| 14-18 | years | 1.3 | 1.0 | 1.4 | 1.6 |
| 19-50 | years | 1.3 | 1.1 | 1.4 | 1.6 |
| 51+ | years | 1.3 | 1.1 | | |

TABLE 6
Recommended Adequate Intake for Infants and Recommended
Dietary Allowances for Iron for Infants (7 to
12 months), Children, and Adults

| | | Males | Females | Pregnant | Lactating |
|---|---|---|---|---|---|
| Age | | (μg/day) | (μg/day) | (μg/day) | (μg/day) |
| Infants | | 0.27 | 0.27 | | |
| 7 to 12 | months | 11 | 11 | | |
| 1 to 3 | years | 7 | 7 | | |
| 4 to 8 | years | 10 | 10 | | |
| 9 to 13 | years | 8 | 8 | | |
| 14 to 18 | years | 11 | 15 | 27 | 10 |
| 19 to 50 | years | 8 | 18 | 27 | 9 |
| 51+ | years | 8 | 8 | | |

TABLE 7
Adequate Intakes (AIs) for Calcium

| | | Males | Females | Pregnant | Lactating |
|---|---|---|---|---|---|
| Age | | (mg/day) | (mg/day) | (mg/day) | (mg/day) |
| Birth to 6 | months | 210 | 210 | | |
| 7 to 12 | months | 270 | 270 | | |
| 1 to 3 | years | 500 | 500 | | |
| 4 to 8 | years | 800 | 800 | | |
| 9 to 13 | years | 1,300 | 1,300 | | |
| 14 to 18 | years | 1,300 | 1,300 | 1,300 | 1,300 |
| 19 to 50 | years | 1,000 | 1,000 | 1,000 | 1,000 |
| 51+ | years | 1,200 | 1,200 | | |

TABLE 8
Adequate Intakes (AIs) for Vitamin D

| | | Males | Females | Pregnant | Lactating |
|---|---|---|---|---|---|
| Age | | (μg/day) | (μg/day) | (μg/day) | (μg/day) |
| Birth to 13 | years | 5 (200 IU) | 5 (200 IU) | | |
| 14 to 18 | years | 5 (200 IU) | 5 (200 IU) | 5 (200 IU) | 5 (200 IU) |
| 19 to 50 | years | 5 (200 IU) | 5 (200 IU) | 5 (200 IU) | 5 (200 IU) |
| 51 to 70 | years | 10 (400 IU) | 10 (400 IU) | | |
| 71+ | years | 15 (600 IU) | 15 (600 IU) | | |

The recommended amount of Other Elements to be administered is from about 30% to about 5000% of its daily recommended allowance, e.g., from 100% to about 2500%, or from about 150% to about 1000% of the daily recommended allowance. Lower amounts may be necessary in preventative cases or premature/neonate cases. While these ranges can be used as a guide, the best practice is for the physician to determine the amount based upon the age, weight and severity of the condition.

To the extent that this invention is treating a fetus, a premature newborn or a term neonate who may also be receiving adequate nutritional supplementation from other sources given such individual's then current medical status, trace amounts of one or more of a folate, vitamin B12, vitamin B6, or vitamin B2 may be sufficient to treat the conditions associated with AHDS. What is important is to determine the total amounts of these vitamins from all of the mother's nutritional intake and the nutritional intake by the newborn immediately after birth in determining the proper amounts to be administered by this embodiment of the invention.

Given that women are carriers of the MCT8 mutation, pregnant woman can pass the MCT8 genetic defect to the fetus. One embodiment of the disclosed subject matter is to administer a pharmaceutical composition or nutritional supplement comprising one or more of a folate, vitamin B12, vitamin B6, or vitamin B2 to these pregnant women for passage through the placental barrier to the fetus. In another embodiment, this administration can also be coupled with the administration of one or more of the Other Elements and/or one or more of the other vitamins of the Vitamin B Complex, which can be administered by any manner already known in the art.

Given that individuals with AHDS can have hyperthyroidism in the blood outside of the CNS, the administration of anti-thyroid drugs may be utilized. An anti-thyroid drug is a hormone antagonist acting upon thyroid hormones. Examples include: propylthiouracil, methimazole, carbimazole, potassium perchlorate, and potassium iodide. Since people taking an anti-thyroid drug are susceptible to developing conditions related to decreased folate levels, one embodiment of this invention provides a composition which comprises an anti-thyroid drug coupled with one or more of a folate, vitamin B12, vitamin B6, or vitamin B2. Administration of these nutrients along with the drug would prevent a folate deficiency from arising or treat a folate deficiency. Propylthiouracil is a common anti-thyroid drug. Propylthiouracil is a thioamide drug used to treat hyperthyroidism (including Graves disease) by decreasing the amount of thyroid hormone produced by the thyroid gland. PTU inhibits the enzyme thyroperoxidase. Propylthiouracil is classified as Drug Class D in pregnancy. Class D signifies that there is positive evidence of human fetal risk. As of 2009, the Food and Drug Administration issued a warning with respect to Propylthiouracil use due to the adverse hepatic damage that it causes. Maternal benefit can outweigh fetal risk in life-threatening situations. The primary effect on the fetus from transplacental passage of PTU is the production of a mild hypothyroidism when the drug is used close to term. This usually resolves within a few days without treatment. The hypothyroid state can be observed as a goiter in the newborn and is the result of increased levels of fetal pituitary thyrotropin. In one embodiment, a composition of propylthiouracil and one or more of a folate, vitamin B12, vitamin B6, or vitamin B2 is created to be administered to people who need to take anti-thyroid drugs. Methimazole is another common anti-thyroid drug. In another embodiment, a composition of methimazole and one or more of a folate, vitamin B12, vitamin B6, or vitamin B2 is created to be administered to people who need to take anti-thyroid drugs. This invention is not limited to the specific anti-thyroid drugs that are mentioned, rather a composition of any anti-thyroid drug can be coupled with one or more of a folate, vitamin B12, vitamin B6, or vitamin B2. In another embodiment, this administration can also be coupled with the administration of one or more of the Other Elements and/or one or more of the other vitamins of the Vitamin B Complex, which can be administered by any manner already known in the art.

### Treatment Methods

Many embodiments of this invention require the administration of folate, or reduced folates. Folates are administered to treat the folate deficiency created by AHDS. In one embodiment, folic acid is the folate that is administered. Folic acid is not biologically active, but it is an effective treatment for many people who have the ability to convert folic acid into its tetrahydrofolate derivatives.

In some instances, folic acid treatment is not enough as folic acid is not the biologically active form of folate. Some individuals have difficulty reducing folic acid into its more biologically active form, therefore, it is necessary to provide these individuals with a reduced folate. A preferred embodiment of the invention is the administration of a reduced folate. It is estimated that administration of a reduced folate is sufficient to prevent and treat a large percentage of people with AHDS. However, a material percentage must still receive 5-methyltetrahydrofolic acid or 5-methyltetrahydrofolate to adequately prevent and/or treat the conditions brought upon by the folate deficiencies due to AHDS. This is the most preferred embodiment of the invention. Indeed, even if an individual's blood levels and cerebrospinal fluid levels of folate are normal, there can still be deficiencies of folate at the intracellular level in brain tissue that require the most biologically active and cell permeable form of folate, which is 5-methyltetrahydrofolate. Further, while in some cases the treatment of folate can be enough to treat the folate deficiencies, in other cases the administration of one or more of vitamin B12, vitamin B6, vitamin B2 and/or one or more of the Other Elements and/or one or more of the other vitamins of the Vitamin B Complex is necessary.

### Compositions

Some of the disclosed compositions comprise folate at an amount effective to treat AHDS. Folate is an essential water-soluble B vitamin that occurs naturally in food. As a result of these important metabolic activities, several dietary derivatives of folate are manufactured as supplements. Although most of the derivatives are capable of becoming converted into the metabolically active form (6S) 5-methyltetrahydrofolate, the enzyme kinetics of such conversion can differ dramatically as well as the absorption rate and it is these differences that are important in determining the hierarchy of performance. As such, L-methylfolate (5-methyltetrahydrofolate or 5-methyltetrahydrofolic acid) and derivatives thereof can be preferred over other reduced folates (including folinic acid) due to its enzyme kinetics and conversion benefits.

Folates are a group of pteroyglutamate acids that become structurally and functionally altered when reduced (adding electrons) or oxidized (removing electrons). In humans, folates are absorbed most readily as 5-methyltetrahydrofolate and it is the principal circulating form of folate. Other derivatives are hydrolyzed in the intestinal jejunum and the liver to the active form with an intermediate stable form (5, 10-methylenetetrahydrofolate). 5-methyltetrahydrofolate is the predominant form of folate in the circulatory system and is the type of folate that can cross the blood-brain barrier and the blood CSF barrier. The metabolism of 5-methyltetrahydrofolate at an intracellular level in brain tissue is instrumental in many metabolic processes that occur in the cell and certain organelles of a cell, and therefore 5-methyltetrahydrofolate is critical for brain development and normal mental health.

In the disclosed compositions, folate can be present at from about 200 mcg to about 15 mg, from about 200 mcg to about 400 mcg, from about 190 mcg to about 390 mcg, from about 210 mcg to about 410 mcg, from about 400 mcg to about 800 mcg, from about 390 mcg to about 790 mcg, from about 410 mcg to about 810 mcg, from about 1.2 mg to about 1.9 mg, from about 2.0 mg to about 2.9 mg, from about 3.0 mg to about 3.9 mg, from about 4.0 mg to about 4.9 mg, from about 5.0 mg to about 5.9 mg, from about 6.0 mg to about 6.9 mg, from about 7.0 mg to about 7.9 mg, from about 8.0 mg to about 8.9 mg, from about 9.0 mg to about 9.9 mg, from about 10.0 mg to about 10.9 mg, from about 11.0 mg to about 11.9 mg, from about 12.0 mg to about 12.9 mg, from about 13.0 mg to about 13.9 mg, or from about 14.0 mg to about 15.0 mg. Still further, the disclosed compositions can contain about 190 mcg, 200 mcg, 210 mcg, 390 mcg, 400 mcg, 410 mcg, 790 mcg, 800 mcg, 810 mcg, 990 mcg, 1 mg, 1.1 mg, 1.2 mg, 1.3 mg, 1.4 mg, 1.5 mg, 1.6 mg, 1.7 mg, 1.8 mg, 1.9 mg, 2.0 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13.0 mg, 13.5 mg, 14.0 mg, 14.5 mg, or 15.0 mg folate, where any of the stated values can form an upper or lower endpoint of a range.

In the disclosed compositions, vitamin B12 can be present at from about 200 mcg to about 3 mg, from about 200 mcg to about 500 mcg, from about 190 mcg to about 490 mcg, from about 210 mcg to about 510 mcg, from about 500 mcg to about 1 mg, from about 490 mcg to about 990 mcg, from about 510 mcg to about 1.1 mg, from about 1 mg to about 1.5 mg, from about 900 mcg to about 1.4 mg, from about 1.1 mg to about 1.6 mg, from about 1.5 mg to about 2.0 mg, from about 1.4 mg to about 1.9 mg, from about 1.6 mg to about 2.1 mg, from about 2.0 mg to about 2.5 mg, from about 1.9 mg to about 2.4 mg, from about 2.1 mg to about 2.6 mg, from about 2.5 mg to about 3.0 mg, from about 2.4 mg to about 2.9 mg, or from about 2.6 mg to about 3.1 mg. Still further, the disclosed compositions can contain about 10 450 mcg, 500 mcg, 550 mcg, 950 mcg, 1 mg, 1.1 mg, 1.4 mg, 1.5 mg, 1.6 mg, 1.9 mg, 2.0 mg, 2.1 mg, 2.4 mg, 2.5 mg, 2.6 mg, 2.9 mg, 3.0 mg, or 3.1 mg of vitamin B12, where any of the stated values can form an upper or lower endpoint of a range.

In the disclosed compositions, vitamin B6 can be present at from about 100 mcg to about 3 mg, from about 200 mcg to about 500 mcg, from about 190 mcg to about 490 mcg, from about 210 mcg to about 510 mcg, from about 500 mcg to about 1 mg, from about 490 mcg to about 990 mcg, from about 510 mcg to about 1.1 mg, from about 1 mg to about 1.5 mg, from about 900 mcg to about 1.4 mg, from about 1.1 mg to about 1.6 mg, from about 1.5 mg to about 2.0 mg, from about 1.4 mg to about 1.9 mg, from about 1.6 mg to about 2.1 mg, from about 2.0 mg to about 2.5 mg, from about 1.9 mg to about 2.4 mg, from about 2.1 mg to about 2.6 mg, from about 2.5 mg to about 3.0 mg, from about 2.4 mg to about 2.9 mg, or from about 2.6 mg to about 3.1 mg. Still further, the disclosed compositions can contain about 100 mcg, 150 mcg, 200 mcg, 300 mcg, 500 mcg, 550 mcg, 950 mcg, 1 mg, 1.1 mg, 1.4 mg, 1.5 mg, 1.6 mg, 1.9 mg, 2.0 mg, 2.1 mg, 2.4 mg, 2.5 mg, 2.6 mg, 2.9 mg, 3.0 mg, or 3.1 mg of vitamin B6, where any of the stated values can form an upper or lower endpoint of a range. Still further the disclosed compositions can contain about 5 mg, 10 mg, 15 mg, 20 mg, 25 mg, 30 mg, 35 mg, 40 mg, 45 mg, 50 mg, 55 mg, 60 mg, or 65 mg of vitamin B6, where any of the stated values can form an upper or lower endpoint of a range. In the disclosed compositions, vitamin B6 can be present at from about 20 mg to about 62 mg. Still further, the disclosed compositions can contain about 22 mg, 27 mg, 32 mg, 37 mg, 42 mg, 47 mg, 52 mg, 57 mg, or 62 mg of vitamin B6, where any of the stated values can form an upper or lower endpoint of a range.

In the disclosed compositions, vitamin B2 can be present at from about 100 mcg to about 3 mg, from about 200 mcg to about 500 mcg, from about 190 mcg to about 490 mcg, from about 210 mcg to about 510 mcg, from about 500 mcg to about 1 mg, from about 490 mcg to about 990 mcg, from about 510 mcg to about 1.1 mg, from about 1 mg to about 1.5 mg, from about 900 mcg to about 1.4 mg, from about 1.1 mg to about 1.6 mg, from about 1.5 mg to about 2.0 mg, from about 1.4 mg to about 1.9 mg, from about 1.6 mg to about 2.1 mg, from about 2.0 mg to about 2.5 mg, from about 1.9 mg to about 2.4 mg, from about 2.1 mg to about 2.6 mg, from about 2.5 mg to about 3.0 mg, from about 2.4 mg to about 2.9 mg, or from about 2.6 mg to about 3.1 mg. Still further, the disclosed compositions can contain about 100 mcg, 150 mcg, 200 mcg, 300 mcg, 500 mcg, 550 mcg, 950 mcg, 1 mg, 1.1 mg, 1.4 mg, 1.5 mg, 1.6 mg, 1.9 mg, 2.0 mg, 2.1 mg, 2.4 mg, 2.5 mg, 2.6 mg, 2.9 mg, 3.0 mg, or 3.1 mg of vitamin B2, where any of the stated values can form an upper or lower endpoint of a range. Still further the disclosed compositions can contain about 5 mg, 10 mg, 15 mg, 20 mg, 25 mg, 30 mg, 35 mg, 40 mg, 45 mg, 50 mg, 55 mg, 60 mg, or 65 mg of vitamin B6, where any of the stated values can form an upper or lower endpoint of a range.

One embodiment disclosed herein is a composition comprising a thyroid hormone analog and one or more of a folate, vitamin B12, vitamin B6, or vitamin B2. The thyroid hormone analog could be any drug that has been approved to treat AHDS. A nonexclusive list includes: diiodothyropropionic acid (DITPA), tetraiodothyroacetic acid (TETRAC), and triiodothyroacetic acid (TRIAC or Tiratricol or Teatrois). Clemastine may be utilized in conjunction with thyroid hormone analogs. The amounts of thyroid hormone analog would be the amounts a physician would prescribe that is appropriate for the patient's condition. The amount of folate can be at least 30% or more of the generally recommended allowance by the NIH, depending on what additional supplements the patient may be taking. The amount of vitamin B12 can be at least 30% or more of the generally recommended allowance by the NIH, depending on what additional supplements the patient may be taking. The amount of vitamin B6 can be at least 30% or more of the generally recommended allowance by the NIH, depending on what additional supplements the patient may be taking. The amount of vitamin B2 can be at least 30% or more of the generally recommended allowance by the NIH, depending on what additional supplements the patient may be taking. Dosage amounts may need to be increased or decreased depending on such factors. This composition may be administered by any means necessary already known in the art. The combination of a thyroid hormone analog and one or more of a folate, vitamin B12, vitamin B6, or vitamin B2 can serve to provide such nutrients to an individual with AHDS and support the metabolic pathways associated with deficiencies related thereto. In a more preferred embodiment of the invention, the folate in the thyroid hormone analog composition would be a reduced folate. The amount of reduced folate can be at least 30% or more of the generally recommended allowance of folic acid by the NIH, depending on what additional supplements the patient may be taking. Dosage amounts may need to be increased or decreased depending on such factors. Since reduced folates are more biologically active, a reduced folate would be more effective in treating folate related deficiencies in brain tissue in the CNS. In a more preferred embodiment of the invention, the folate in the thyroid hormone analog composition would be 5-methyltetrahydrofolic acid or 5-methyltetrahydrofolate. The amount of 5-methyltetrahydrofolic acid or 5-methyltetrahydrofolate can be at least 30% or more of the generally recommended allowance for folic acid by the NIH. Depending on what additional supplements the patient may be taking, dosage amounts may need to be increased or decreased depending on such factors. In other embodiments of this invention, one or more of Other Elements and/or one or more of vitamins of the Vitamin B Complex would be utilized in a composition as disclosed herein. For example, a composition comprising one or more of a folate, vitamin B12, vitamin B6, or vitamin B2, can be in combination with the thyroid hormone analog, and one or more of the Other Elements and/or one or more of vitamins of the Vitamin B Complex. In another example, a composition can comprise the thyroid hormone analog and just one or more of the Other Elements and/or one or more of vitamins of the Vitamin B Complex.

One embodiment disclosed herein is a composition comprising a thyroid hormone chaperone and one or more of a folate, vitamin B12, vitamin B6, or vitamin B2. The thyroid hormone chaperone could be any drug that has been approved to treat AHDS. A nonexclusive list includes: phenylbutyrate, 4-phenylbutyric acid, and sodium phenylbutyrate. The amounts of thyroid hormone chaperone would be the amounts a physician would prescribe that is appropriate for the patient's condition. The amount of folate can be at least 30% or more of the generally recommended allowance by the NIH, depending on what additional supplements the patient may be taking. The amount of vitamin B12 can be at least 30% or more of the generally recommended allowance by the NIH, depending on what additional supplements the patient may be taking. The amount of vitamin B6 can be at least 30% or more of the generally recommended allowance by the NIH, depending on what additional supplements the patient may be taking. The amount of vitamin B2 can be at least 30% or more of the generally recommended allowance by the NIH, depending on what additional supplements the patient may be taking. Dosage amounts may need to be increased or decreased depending on such factors. This composition may be administered by any means necessary already known in the art. The combination of a thyroid hormone chaperone and one or more of a folate, vitamin B12, vitamin B6, or vitamin B2 can serve to provide such nutrients to an individual with AHDS and support the metabolic pathways associated with deficiencies related thereto. In a more preferred embodiment of the invention, the folate in the thyroid hormone chaperone composition would be a reduced folate. The amount of reduced folate can be at least 30% or more of the generally recommended allowance of folic acid by the NIH, depending on what additional supplements the patient may be taking. Dosage amounts may need to be increased or decreased depending on such factors. Since reduced folates are more biologically active, a reduced folate would be more effective in treating folate related deficiencies in brain tissue in the CNS. In a more preferred embodiment of the invention, the folate in the thyroid hormone chaperone composition would be 5-methyltetrahydrofolic acid or 5-methyltetrahydrofolate. The amount of 5-methyltetrahydrofolic acid or 5-methyltetrahydrofolate can be at least 30% or more of the generally recommended allowance for folic acid by the NIH. Depending on what additional supplements the patient may be taking, dosage amounts may need to be increased or decreased depending on such factors. In other embodiments of this invention, one or more of Other Elements or vitamins of the Vitamin B Complex would be utilized. For example, a composition comprising one or more of a folate, vitamin B12, vitamin B6, or vitamin B2, can be in combination with the thyroid hormone chaperone, and one or more of the Other Elements and/or one or more of vitamins of the Vitamin B Complex. In another example, a composition can comprise the thyroid hormone chaperone and just one or more of the Other Elements and/or one or more of vitamins of the Vitamin B Complex.

One embodiment disclosed herein is a composition comprising a thyroid hormone thyromimetic and one or more of a folate, vitamin B12, vitamin B6, or vitamin B2. The thyroid hormone thyromimetic could be any drug that has been approved to treat AHDS. A nonexclusive list includes: eprotirome, sobetirome, and Sob-AM2. The amounts of thyroid hormone thyromimetic would be the amounts a physician would prescribe that is appropriate for the patient's condition. The amount of folate can be at least 30% or more of the generally recommended allowance by the NIH, depending on what additional supplements the patient may be taking. The amount of vitamin B12 can be at least 30% or more of the generally recommended allowance by the NIH, depending on what additional supplements the patient may be taking. The amount of vitamin B6 can be at least 30% or more of the generally recommended allowance by the NIH, depending on what additional supplements the patient may be taking. The amount of vitamin B2 can be at least 30% or more of the generally recommended allowance by the NIH, depending on what additional supplements the patient may be taking. Dosage amounts may need to be increased or decreased depending on such factors. This composition may be administered by any means necessary already known in the art. The combination of a thyroid hormone thyromimetic and one or more of a folate, vitamin B12, vitamin B6, or vitamin B2 can serve to provide such nutrients to an individual with AHDS and support the metabolic pathways associated with deficiencies related thereto. In a more preferred embodiment of the invention, the folate in the thyroid hormone thyromimetic composition would be a reduced folate. The amount of reduced folate can be at least 30% or more of the generally recommended allowance of folic acid by the NIH, depending on what additional supplements the patient may be taking. Dosage amounts may need to be increased or decreased depending on such factors. Since reduced folates are more biologically active, a reduced folate would be more effective in treating folate related deficiencies in brain tissue in the CNS. In a more preferred embodiment of the invention, the folate in the thyroid hormone thyromimetic composition would be 5-methyltetrahydrofolic acid or 5-methyltetrahydrofolate. The amount of 5-methyltetrahydrofolic acid or 5-methyltetrahydrofolate can be at least 30% or more of the generally recommended allowance for folic acid by the NIH. Depending on what additional supplements the patient may be taking, dosage amounts may need to be increased or decreased depending on such factors. In other embodiments of this invention, one or more of Other Elements or vitamins of the Vitamin B Complex would be utilized. For example, a composition comprising one or more of a folate, vitamin B12, vitamin B6, or vitamin B2, can be in combination with the thyroid hormone thyromimetic composition, and one or more of the Other Elements and/or one or more of vitamins of the Vitamin B Complex. In another example, a composition can comprise the thyroid hormone thyromimetic composition and just one or more of the Other Elements and/or one or more of vitamins of the Vitamin B Complex.

One embodiment disclosed herein is a composition comprising a thyroid hormone gene therapy and one or more of a folate, vitamin B12, vitamin B6, or vitamin B2. The thyroid hormone gene therapy could be any drug or method that has been approved to treat AHDS. A nonexclusive list includes: adeno associated virus 9 based gene therapy (AAV9). The amounts of or method of delivering a thyroid hormone gene therapy would be the amounts or methods a physician would prescribe that is appropriate for the patient's condition. The amount of folate can be at least 30% or more of the generally recommended allowance by the NIH, depending on what additional supplements the patient may be taking. The amount of vitamin B12 can be at least 30% or more of the generally recommended allowance by the NIH, depending on what additional supplements the patient may be taking. The amount of vitamin B6 can be at least 30% or more of the generally recommended allowance by the NIH, depending on what additional supplements the patient may be taking. The amount of vitamin B2 can be at least 30% or more of the generally recommended allowance by the NIH, depending on what additional supplements the patient may be taking. Dosage amounts may need to be increased or decreased depending on such factors. This composition may be administered by any means necessary already known in the art. The combination of a thyroid hormone gene therapy and one or more of a folate, vitamin B12, vitamin B6, or vitamin B2 can serve to provide such nutrients to an individual with AHDS and support the metabolic pathways associated with deficiencies related thereto. In a more preferred embodiment of the invention, the folate in the thyroid hormone gene therapy composition would be a reduced folate. The amount of reduced folate can be at least 30% or more of the generally recommended allowance of folic acid by the NIH, depending on what additional supplements the patient may be taking. Dosage amounts may need to be increased or decreased depending on such factors. Since reduced folates are more biologically active, a reduced folate would be more effective in treating folate related deficiencies in brain tissue in the CNS. In a more preferred embodiment of the invention, the folate in the thyroid hormone gene therapy composition would be 5-methyltetrahydrofolic acid or 5-methyltetrahydrofolate. The amount of 5-methyltetrahydrofolic acid or 5-methyltetrahydrofolate can be at least 30% or more of the generally recommended allowance for folic acid by the NIH. Depending on what additional supplements the patient may be taking, dosage amounts may need to be increased or decreased depending on such factors. In other embodiments of this invention, one or more of Other Elements or vitamins of the Vitamin B Complex would be utilized. For example, a composition comprising one or more of a folate, vitamin B12, vitamin B6, or vitamin B2, can be in combination with the thyroid hormone gene therapy, and one or more of the Other Elements and/or one or more of vitamins of the Vitamin B Complex. In another example, a composition can comprise the thyroid hormone gene therapy and just one or more of the Other Elements and/or one or more of vitamins of the Vitamin B Complex.

One embodiment disclosed herein is a composition comprising an anti-thyroid drug and one or more of the Other Elements and/or one or more vitamins of the Vitamin B Complex. The anti-thyroid drugs could be any drug that has been approved to treat an overactive thyroid gland or suppress thyroid function or decrease excessive thyroid hormone. A nonexclusive list includes: propylthiouracil, methimazole, carbimazole, potassium perchlorate, and potassium iodide. The amounts of anti-thyroid drug would be the amounts a physician would prescribe that is appropriate for the patient's condition. The amount of Other Elements and/or vitamins of the Vitamin B Complex can be an amount sufficient to alleviate the patient's symptoms, which can be determined by increasing dosages until a desired result is obtained.

In another embodiment disclosed herein, there is a composition comprising a thyroid hormone drug and one or more of the Other Elements and/or one or more vitamins of the Vitamin B Complex. The thyroid hormone drug could be any drug or hormone that has been approved to treat underactive thyroid function or that is a natural thyroid replacement therapy such as desiccated thyroid hormone, and that is not otherwise a thyroid hormone analog, thyroid hormone chaperone, thyroid hormone thyromimetic, or thyroid hormone gene therapy. A nonexclusive list includes: Levothyroxine, Levothyroxine Sodium, Liothyronine Sodium, Liotrix, Thyroglobulin, Thyroid (for example, desiccated thyroid hormone), Thyroxine, Triiodothyronine, and may be sold under the brand names Levoxyl, Synthroid, Levo-T, Unithroid, Levothroid, Levoxine, Levolet, Novothyrox, Triostat, Cytomel and Thyrolar. The definition of “thyroid hormone drug” also includes combinations of the foregoing such as T4/T3 blends for instance, as well as slow release, controlled release, delayed release, or similar versions of the foregoing. The amounts of thyroid hormone drug would be the amounts a physician would prescribe that is appropriate for the patient's condition. The amount of Other Elements and/or vitamins of the Vitamin B Complex can be an amount sufficient to alleviate the patient's symptoms, which can be determined by increasing dosages until a desired result is obtained.

### Delivery Methods

In vivo application of the disclosed compositions can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection.

Administration of the disclosed compounds or compositions can be a single administration, or at continuous or distinct intervals as can be readily determined by a person skilled in the art.

The compositions disclosed herein can also be administered utilizing liposome technology, controlled release capsules, tablets, pills, and implants, implantable pumps, and biodegradable containers. These delivery methods can, advantageously, provide a uniform dosage over an extended period of time. There is a need to have a controlled release composition when using folate, vitamin B12, vitamin B6, and/or vitamin B2 in patients taking thyroid hormone drugs such that the folate, vitamin B2, B6, and/or B2 is released 4-6 hrs after the thyroid drug is released. In a combined pill that first releases the thyroid drug consistent with its normal absorption profile and then 4-6 hrs. later, the folate, vitamin B2, B6, and/or B2 is released. There is also a needed to have controlled release compositions comprising one or more of the Other Elements or one or more of the vitamins of the Vitamin B Complex.

The compounds can also be administered in their salt derivative forms or crystalline forms.

The compositions disclosed herein can be formulated according to known methods for preparing pharmaceutically acceptable compositions. Formulations are described in detail in a number of sources which are well known and readily available to those skilled in the art. For example, Remington's Pharmaceutical Science by E.W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question Therapeutic application of the disclosed compositions can be accomplished by any suitable therapeutic method and technique presently or prospectively known to those skilled in the art. Further, compositions disclosed herein have use as starting materials or intermediates for the preparation of other useful compounds and compositions.

Compositions disclosed herein can be locally administered at one or more anatomical sites, injected or topically applied, optionally in combination with a pharmaceutically acceptable carrier such as an inert diluent. Compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compositions disclosed herein, including pharmaceutically acceptable salts, hydrates, or analogs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze-drying techniques, which yield a powder of the active ingredient plus any additional desired ingredient present in the previously sterile-filtered solutions. For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like. Drug delivery systems for delivery of pharmacological substances to dermal lesions can also be used, such as that described in U.S. Pat. No. 5,167,649.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver a compound to the skin are disclosed in U.S. Pat. Nos. 4,608,392; 4,992,478; 4,559,157; and 4,820,508. Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art; for example, see U.S. Pat. No. 4,938,949.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

### Dosing Frequency

Dosing frequency for the disclosed compositions includes, but is not limited to, at least about once every 7 days, once every 6 days, once every 5 days, once every 4 days, once every 3 days, once every 2 days, or daily. In some embodiments, the interval between each administration is less than about a week, such as less than about any of 6, 5, 4, 3, 2, or 1 day. In some embodiments, the dosing frequency for the disclosed compositions includes, but is not limited to, at least once a day, twice a day, three times a day, or four times a day. In some embodiments, the interval between each administration is less than about 48 hours, 36 hours, 24 hours, 22 hours, 20 hours, 18 hours, 16 hours, 14 hours, 12 hours, 10 hours, 9 hours, 8 hours, 7 hours, 6 hours, or 5 hours. In some embodiments, the interval between each administration is less than about 24 hours, 22 hours, 20 hours, 18 hours, 16 hours, 14 hours, 12 hours, 10 hours, 9 hours, 8 hours, 7 hours, 6 hours, or 5 hours. In some embodiments, the interval between each administration is constant. Administration can also be continuous and adjusted to maintaining a level of the compound within any desired and specified range.

The administration of the disclosed composition can be extended over an extended period of time, such as from about a month or shorter up to about three years or longer. For example, the dosing regimen can be extended over a period of any of about 2, 3, 4, 5, 6, 7, 8, 9, 10, 11, 12, 18, 24, 30, and 36 months. In some embodiments, there is no break in the dosing schedule. In some embodiments, the interval between a course of administration is no more than about a week.

### Examples

The following examples illustrate the medical conditions presented in individuals with AHDS and the confounding nature of their laboratory and diagnostic testing results. As noted in Tonduti D, Vanderver A, Berardinelli A, et al. MCT8 Deficiency: Extrapyramidal Symptoms and Delayed Myelination as Prominent Features. J. Child Neurol. 2013; 28(6): 795-800, which is incorporated by reference herein in its entirety, a case study on Patient 3 who had AHDS was described (“Case Study #3”). As a result of having AHDS, Case Study #3 by virtue of AHDS had profound hypothyroidism in utero in his CNS in his brain tissue, yet his thyroid function tests for his blood (outside of the CNS) demonstrated a normal free T3 level, a relatively low free T4 level (3.4 pmol/L, normal values 5.9-11.6 pmol/L), and normal TSH levels. Indeed, Case Study #3 presented with classic signs of AHDS, with a relative euthyroid clinical picture in the blood outside of the CNS, but (unknowingly to practitioners) profound hypothyroidism in the CNS in his brain tissue. Sometimes in AHDS, individuals even show a clinical picture of hyperthyroidism in the blood (outside of the CNS), while simultaneously having profound hypothyroidism in the CNS in brain tissue (see case reports with respect to Patient 1 and Patient 2 in the Tonduti article who each had high levels of free T3 in the blood, indicative of a more hyperthyroid clinical picture, while simultaneously having profound hypothyroidism in the CNS in brain tissue).

Case Study #3 presented at 3 months of age with increased tone and developmental delay. Applicant notes, in Applicant's earlier application, WO 2011/006147 A1, that the Example #2 referenced therein also displayed manifestations of tone issues and developmental delay at approximately the same age, after suffering from profound hypothyroidism in utero as well similar to that of Case Study #3, yet Example #2's hypothyroidism emanated from the blood (outside of the CNS) rather than emanating from the CNS as in the case of Case Study #3. Example #2's profound hypothyroidism was brought upon in utero by virtue of Example #2's mother being overmedicated with an anti-thyroid drug. Example #2's profound hypothyroidism continued immediately after birth until Example #2 was rendered chemically euthyroid by virtue of the administration of levothyroxine. Case Study #3 progressively developed a clinical picture characterized by spastic-dystonia, facial grimaces, persistent neonatal reflexes, and cognitive delay. Similarly, Example #2 displayed the same clinical manifestations and progression, despite Example #2 being rendered chemically euthyroid and no longer having a hypothyroid clinical picture in the blood, and by virtue of that, also no longer having a clinical picture of hypothyroidism in the CNS. However, as noted in WO 2011/006147 A1, Example #2 was eventually diagnosed with cerebrospinal folate deficiency at 5.25 years old, because the residual folate deficiencies that the earlier hypothyroidism caused in Example #2's cerebrospinal fluid still remained. After the cerebrospinal folate deficiency was identified, and Example #2 received a reduced folate, Example #2 promptly responded, ultimately with resolution of Example #2's tone, developmental delay, spastic-dystonia, facial grimaces, reflex issues, and cognitive delay.

Although the Tonduti article does not identify the precise time Case Study #3 underwent cerebrospinal fluid testing, the Tonduti article does indicate that testing was performed at 4 years old or younger. At such time, a multitude of cerebrospinal fluid testing was performed on Case Study #3, including folate in cerebrospinal fluid. However, Case Study #3's cerebrospinal fluid testing, including folate, was normal. These results even further confound the clinical picture of an individual with AHDS. With respect to Case Study #3, Case Study #3 presented with relative euthyroidism in the blood, thereby not leading a practitioner to expect profound hypothyroidism in the CNS in brain tissue. Further, even when cerebrospinal fluid testing was performed to identify abnormalities in cerebrospinal fluid, those test results came back normal.

Applicant has, however, identified individuals with AHDS who are in their adolescent years who indeed have deficiencies of folate in their cerebrospinal fluid. This is because in an AHDS individual's younger years, the folate deficiencies that develop intracellularly in the CNS in brain tissue have not reached a point of reflection in the cerebrospinal fluid. But, as an individual with AHDS increases in age, and as the intracellular folate needs of the cells of brain tissue reach a stage of overconsumption of folate, deficiencies in folate in the cerebrospinal fluid can arise. Unfortunately, from diagnostic and laboratory testing perspectives, it may indeed be too late given that due to the debilitating nature of AHDS at a very young age, most diagnostic and laboratory testing and interventions are conducted at an early age, wherein a deficiency in folate in the cerebrospinal fluid may not have manifested. When cerebrospinal fluid testing results come back with a normal folate result, a practitioner will rule that deficiency out and move on to other diagnostic and laboratory approaches and interventions; thus, the need for the methods and compositions taught by Applicant.

Example #2 exhibited many of the same manifestations and progression of symptoms as individuals with AHDS. In classic cerebral folate deficiency, many of such individuals exhibit many of the same manifestations and progression of symptoms as individuals with AHDS as well. In the case of Example #2, and in the case of individuals with classic cerebral folate deficiency, folate deficiencies in cerebrospinal fluid are evident. Reduced folate treatment resolved Example #2's symptoms. In classic cerebral folate deficiency, reduced folate resolves or substantially alleviates the symptoms those individuals suffer from. Given the deficiencies in folate in the CNS in brain tissue of AHDS individuals, they too are in need of the methods and compositions taught by Applicant herein.

### SPECIFIC EMBODIMENTS

Disclosed herein, in certain embodiment, is a method of treating an individual with a monocarboxylate transporter 8 (MCT8) deficiency (Allan-Herndon-Dudley Syndrome), comprising: administering to the individual a composition comprising 5-methyltetrahydrofolate or 5-methyltetrahydrofolic acid. The administered composition can be a pharmaceutical composition or nutritional supplement. Further the method can include the administration of vitamin B6, e.g., the vitamin B6 can be selected from the group consisting of pyridoxal, pyridoxol, pyridoxine, pyridoxamine, pyridoxamine phosphate, and pyridoxine hydrochloride. In specific examples, the vitamin B6 is pyridoxal 5′-phosphate. In further examples, the method can further comprise the administration of vitamin B2, e.g., wherein the vitamin B2 is selected from the group consisting of riboflavin, flavin mononucleotide (FMN), flavin adenine dinucleotide (FAD), and dihydro-flavin adenine dinucleotide (FADH₂).

In further examples, the method can further comprise the administration of a thyroid hormone analog, thyroid hormone chaperone, thyroid hormone thyromimetic, or thyroid hormone gene therapy.

In further examples, the method can further comprise the administration of a thyroid hormone analog, thyroid hormone chaperone, thyroid hormone thyromimetic, or thyroid hormone gene therapy, wherein the thyroid hormone analog, thyroid hormone chaperone, thyroid hormone thyromimetic, or thyroid hormone gene therapy is selected from the group consisting of diiodothyropropionic acid (DITPA), tetraiodothyroacetic acid (TETRAC), triiodothyroacetic acid (TRIAC or Tiratricol or Teatrois), phenylbutyrate, 4-phenylbutyric acid, sodium phenylbutyrate, adeno associated virus 9 based gene therapy (AAV9), eprotirome, sobetirome, or Sob-AM2.

In other embodiments, disclosed is a composition comprising (a) a thyroid hormone analog, and (b) 5-methyltetrahydrofolate or 5-methyltetrahydrofolic acid. In specific examples, the thyroid hormone analog can be diiodothyropropionic acid (DITPA) or triiodothyroacetic acid (TRIAC or Tiratricol or Teatrois).

In other examples, the composition can further comprise vitamin B6, e.g., wherein the vitamin B6 is selected from the group consisting of pyridoxal, pyridoxol, and pyridoxine, more specifically, the vitamin B6 is pyridoxal 5′-phosphate.

In other examples, the composition can further comprise vitamin B2, e.g., where the vitamin B2 is selected from the group consisting of riboflavin, flavin mononucleotide (FMN), flavin adenine dinucleotide (FAD), and dihydro-flavin adenine dinucleotide (FADH₂).

1. A method of treating an individual with a monocarboxylate transporter 8 (MCT8) deficiency (Allan-Herndon-Dudley Syndrome), comprising: administering to the individual a composition comprising 5-methyltetrahydrofolate or 5-methyltetrahydrofolic acid.

2. The method of claim 1, further comprising the administration of vitamin B6.

3. The method of claim 2, wherein the vitamin B6 is selected from the group consisting of pyridoxal, pyridoxol, pyridoxine, pyridoxamine, pyridoxamine phosphate, and pyridoxine hydrochloride.

4. The method of claim 2, wherein the vitamin B6 is pyridoxal 5′-phosphate.

5. The method of claim 1, further comprising the administration of vitamin B2.

6. The method of claim 5, wherein the vitamin B2 is selected from the group consisting of riboflavin, flavin mononucleotide (FMN), flavin adenine dinucleotide (FAD), and dihydro-flavin adenine dinucleotide (FADH₂).

7. The method of claim 1, further comprising the administration of a thyroid hormone analog, thyroid hormone chaperone, thyroid hormone thyromimetic, or thyroid hormone gene therapy.

8. The method of claim 1, further comprising the administration of a thyroid hormone analog, thyroid hormone chaperone, thyroid hormone thyromimetic, or thyroid hormone gene therapy, wherein the thyroid hormone analog, thyroid hormone chaperone, thyroid hormone thyromimetic, or thyroid hormone gene therapy is selected from the group consisting of diiodothyropropionic acid (DITPA), tetraiodothyroacetic acid (TETRAC), triiodothyroacetic acid (TRIAC or Tiratricol or Teatrois), phenylbutyrate, 4-phenylbutyric acid, sodium phenylbutyrate, adeno associated virus 9 based gene therapy (AAV9), eprotirome, sobetirome, or Sob-AM2.

9. A composition comprising (a) a thyroid hormone analog, and (b) 5-methyltetrahydrofolate or 5-methyltetrahydrofolic acid.

10. The composition of claim 9, wherein the thyroid hormone analog is diiodothyropropionic acid (DITPA) or triiodothyroacetic acid (TRIAC or Tiratricol or Teatrois).

11. The composition of claim 9, further comprising vitamin B6.

12. The composition of claim 11, wherein the vitamin B6 is selected from the group consisting of pyridoxal, pyridoxol, and pyridoxine.

13. The composition of claim 11, wherein the vitamin B6 is pyridoxal 5′-phosphate.

14. The composition of claim 9, further comprising vitamin B2.

15. The composition of claim 14, wherein the vitamin B2 is selected from the group consisting of riboflavin, flavin mononucleotide (FMN), flavin adenine dinucleotide (FAD), and dihydro-flavin adenine dinucleotide (FADH₂).
